(19) United States
(12) Patent Application Publication
(10) Pub. No.: US 2009/0263838 A1
(43) Pub. Date: Oct. 22, 2009

(54) Method for determining a lung cancer treatment and method for determining the effectiveness of an agent for treatment of lung cancer

(75) Inventors: Mitsune Tanimoto, Okayama (JP); Katsuyuki Kiura, Okayama (JP); Yoshiro Fujiwara, Okayama (JP); Shinichi Toyooka, Okayama (JP); Nagio Takigawa, Okayama (JP); Katsuyuki Hotta, Okayama (JP)

Correspondence Address: TAIYO CORPORATION, 401 HOLLAND LANE, #407, ALEXANDRIA, VA, 22314, US

(73) Assignee: Sanko Junyaku Co., Ltd., Chiyoda-ku (JP)

(21) Appl. No.: 12/155,148

(22) Filed: May 30, 2008

Related U.S. Application Data
(60) Provisional application No. 61/071,290, filed on Apr. 21, 2008.

(51) Int. Cl. G01N 33/53

(52) U.S. Cl. 435/7.92; 435/7.1

(57) ABSTRACT

The present invention provides a method of determining a lung cancer treatment, the method comprising: measuring an amount of KL-6 in a sample of body fluid from a subject, and determining the treatment based on the amount of KL-6 measured, and a method of determining the effectiveness of an agent for treatment of lung cancer on a subject, the method comprising: measuring an amount of KL-6 in a sample of body fluid from the subject, determining the effectiveness of an EGFR inhibitor on the subject should the EGFR inhibitor be administered as the agent for treatment of lung cancer, based on the amount of KL-6 measured.

### CROSS-REFERENCE TO RELATED APPLICATIONS

This application claims priority under 35 U.S.C. 119(e) to Provisional U.S. Patent Application No. 61/071,290, filed Apr. 21, 2008, the disclosure of which is incorporated by reference herein.

### BACKGROUND OF THE INVENTION

1. Field of the Invention

The present invention relates to a method for determining a lung cancer treatment and a method for determining the effectiveness of an agent for treatment of lung cancer.

2. Description of the Related Art

Epidermal growth factor receptors (EGFR) are a promising target for cancer therapy, because the signaling thereof drives the proliferation and survival of cancer cells and they have been shown to be frequently overexpressed in various tumor types including non-small cell lung cancer (NSCLC). In fact, gefitinib, an EGFR tyrosine kinase inhibitor, has shown substantial antitumor activity in early phase II clinical trials for patients with advanced NSCLC who had previously received platinum-based chemotherapy [1, 2]. Although a subsequent large randomized phase III trial for pretreated NSCLC failed to demonstrate a significant survival advantage of gefitinib over a placebo in the entire cohort, a subgroup analysis revealed the superiority of gefitinib over a placebo in patients of Asian origin [3]. Recent prospective studies of NSCLC also demonstrated that gefitinib is highly effective in female patients, those with an adenocarcinoma histology, and those without a smoking history [4, 5]. All of these findings suggest the importance of predictors for selecting patients who are most likely to benefit from gefitinib.

Recently, mutations of EGFR tyrosine kinase domain have been identified as a molecular predictor for the treatment outcome of gefitinib [6-9]. Approximately 90% of EGFR mutations are either in-frame deletion mutations in exon 19 or point mutations in exon 21, and they are closely related to known clinical predictors as well as to the response to gefitinib [10]. Research from other groups has identified candidate gene alternations other than EGFR mutations that may be involved in determining the response to gefitinib, including an increased EGFR gene copy number and K-ras mutations [11-13]. However, these genetic markers seem to be somewhat inconvenient to use in daily practice, and not all NSCLC patients are always able to undergo a routine assessment of their gene status in their tumors before starting gefitinib treatment due to technical or financial restraints. Moreover, there can be some interinstitutional variances in the accuracy of the assays [14]. Therefore, new biomarkers appear to be needed that are more readily available in daily clinical practice with the development of the specificity and sensitivity of EGFR mutations.

### SUMMARY OF THE INVENTION

The present invention has been made in view of the above circumstance and provides a method of determining a lung cancer treatment and a method of determining the effectiveness of an agent for treatment of lung cancer on a subject.

A first aspect of the present invention provides a method of determining a lung cancer treatment, the method comprising: measuring an amount of KL-6 in a sample of body fluid from a subject, and determining the treatment based on the amount of KL-6 measured.

A second aspect of the present invention provides a method of determining the effectiveness of an agent for treatment of lung cancer on a subject, the method comprising: measuring an amount of KL-6 in a sample of body fluid from the subject, determining the effectiveness of an EGFR inhibitor on the subject should the EGFR inhibitor be administered as the agent for treatment of lung cancer, based on the amount of KL-6 measured.

### BRIEF DESCRIPTION OF THE DRAWINGS

FIG. 1 is a Kaplan-Meier progression-free survival curve for patients with non-small cell lung cancer who were treated with gefitinib based on serum KL-6 levels. Bold and thin lines represent survival in patients with and without elevated KL-6, respectively (median progression-free survival time; 2.1 versus 5.8 months, log-rank test; p=0.0220).

FIG. 2 is a Kaplan-Meier overall survival curve for patients with non-small cell lung cancer who were treated with gefitinib based on serum KL-6 levels. Bold and thin lines represent survival in patients with and without elevated KL-6, respectively (median overall survival time; 8.5 versus 21.1 months, log-rank test; p=0.0001).

FIG. 3 is a Kaplan-Meier progression-free survival curve for patients with non-small cell lung cancer who were treated with gefitinib based on serum KL-6 levels and EGFR mutation status. Bold, thin, and dotted lines represent survival in patients with wild-type EGFR and elevated KL-6, with wild-type EGFR and normal range of KL-6, and with EGFR mutation, respectively (median overall survival time; 6.5, 13.3, and 17.8 months, respectively).

### DETAILED DESCRIPTION OF THE INVENTION

The present invention is based on an investigation of the prognostic value of KL-6 levels in advanced cancer patients treated with an EGFR inhibitor and consequent determination of the association of KL-6 levels with EGFR mutation status. The factors affecting patient survival after administration of an agent for lung cancer treatment such as gefitinib treatment in patients with lung cancer have yet to be fully elucidated, although EGFR mutation is a substantial prognostic factor. KL-6 has been studied as a useful indicator for interstitial lung diseases; however, it was first discovered as a lung cancer-related antigen. That is, the KL-6 levels in the body fluid might thus be able to provide predictive information about patient survival in the treatment of lung cancer by an agent for treatment of lung cancer and, especially in a preferred embodiment, treatment of NSCLC by gefitinib. In the present invention, it was found that pretreatment KL-6 levels could be used to predict survival in NSCLC patients treated with an agent for treatment of lung cancer, and also to determine the association of KL-6 levels with the EGFR mutation status.

In the present invention, the term “step” is not only used for a discrete step, but also for a step that is indistinguishable from other steps, so long as the intended purpose or results can be accomplished or obtained.

A first embodiment of the present invention provides a method of determining a lung cancer treatment, the method comprising: measuring an amount of KL-6 in a sample of body fluid from a subject (measuring step), and determining the treatment based on the amount of KL-6 measured (determining step).

KL-6 is a high molecular weight (about 200 kD) sialylated glycoprotein that is predominantly expressed on pulmonary epithelial cells [15]. Although KL-6 has been studied as a useful indicator for interstitial lung diseases, it was originally discovered as a lung cancer-related antigen [16]. In addition, the biochemical properties of KL-6 are similar to those of other MUC1 mucins that have been reported to be associated with a poor prognosis in NSCLC [17, 18].

In the present invention, the treatment may include the use of an EGFR inhibitor as an agent for treatment of lung cancer, and the EGFR inhibitor may be any agent that inhibits the functions of an EGFR. Examples of the EGFR inhibitor include EGFR tyrosine kinase inhibitors, anti-EGFR antibodies and the like. Examples of the EGFR tyrosine kinase inhibitor include gefitinib, Erlotinib and the like. In a preferred embodiment of the invention, gefitinib may be used as an EGFR inhibitor and, in particular, as an EGFR tyrosine kinase inhibitor.

In the present invention, the lung cancer may be any kind of lung cancer with respect to which a pharmaceutical effect can be obtained by using an EGFR inhibitor. Small cell lung cancer, squamous cell cancer, adenocarcinoma and large cell cancer are known lung cancers. In a preferred embodiment of the present invention, the lung cancer is non-small cell lung cancer (NSCLC).

In the measuring step of the method of determining a lung cancer treatment of the present invention, the amount of KL-6 in a sample of body fluid from a subject is measured.

The body fluid sample is preferably, but is not limited to, at least one selected from blood, serum, plasma and lymphatic fluid, and serum. The preparation of the sample of body fluid is not particularly limited and can be carried out by conventional methods.

The method for measuring the KL-6 is not limited so long as the amount of KL-6 in a sample of body fluid can be measured. Ordinary methods for detecting proteins may be used, such as a method using anti-KL-6 antibodies, ion exchange chromatography, mass spectrometry and the like. A measuring method using anti-KL-6 antibodies is preferable in view of detection sensitivity, detection specificity and convenience of detection. Anti-KL-6 antibodies are commercially available as a component of kits for enzyme-linked immunosorbent assay (ELISA) or electrochemiluminescence immunoassay (ECL) for detecting KL-6 such as Picolumi KL-6 or Eitest KL-6, manufactured by Sanko Junyaku, Tokyo, Japan.

In a preferred embodiment of the present invention, measurement of the KL-6 level may be performed by immunoassay including the use of anti-KL-6 antibodies and labeled antibodies.

The immunoassay may be carried out by any conventional assay without any particular limitation. Example of the immunoassay includes enzyme-linked immunosorbent assay (ELISA), chemiluminescence immunoassay, electrochemiluminescence immunoassay (ECL), absorption assay, a fluorescent antibody technique, radioimmunoassay (RIA), surface plasmon resonance, Western blotting, dot blotting and the like. In the present invention, enzyme-linked immunosorbent assay (ELISA) or electrochemiluminescence immunoassay (ECL) are preferable in view of detection sensitivity, detection specificity and convenience of detection. Kits for ELISA or ECL for detecting KL-6 are commercially available as, for example, Picolumi KL-6 or Eitest KL-6, manufactured by Sanko Junyaku, Tokyo, Japan.

Any known labeling agent (“a label”) may used to label antibodies and materials in the embodiments of the present invention without any particular limitation. Examples of the label include enzymes, chemiluminescent materials, electrochemiluminescent materials, radioactive materials and the like. In the present invention, enzymes are preferable as a label in view of detection sensitivity and convenience of detection.

The enzymes may be used without any particular limitation so long as quantitative detection can be carried out by a physical or chemical procedure. Examples of the enzymes include alkaline phosphatase, horseradish peroxidase (HRP), luciferase and the like.

The method of detecting the labeled materials is not limited so long as the method is normally used for this purpose. Methods of detecting the labeled materials include, for example, absorbance spectrum, luminescence intensity, fluorescence intensity, or radiation measurement and the like. The method can be selected appropriately in accordance with the kind of label.

In a determining step of the present invention, a lung cancer treatment may be determined based on the amount of KL-6 measured and, for example, an EGFR inhibitor may be selected as the agent for treatment of lung cancer.

For example, when the amount of KL-6 in the body fluid sample of the subject is equal to or higher than a reference level set in order to distinguish between a healthy person and a patient with lung cancer, it can be determined that an EGFR inhibitor will not be effective as an agent for treatment of lung cancer on the patient, and it can be decided that the treatment including use of an EGFR inhibitor on the patient with lung cancer will not be carried out. On the other hand, when the amount of KL-6 of a subject is lower than the reference level, it can be determined that an EGFR inhibitor will be effective as an agent for treatment of lung cancer on the patient, and it can be decided that treatment including use of an EGFR inhibitor on the patient with lung cancer may be carried out.

The reference level used in the determining step may be a level based on any criterion so long as it can used to distinguish between a healthy person and a patient with lung cancer. A healthy person as a control is an individual who has been determined in advance not to have any lung cancer, and not to have NSCLC in particular.

In a preferred embodiment of the present invention, the reference level may be a normal detection level (cut-off level) or an amount of KL-6 of a body fluid sample of another healthy person. A cut-off level can be determined as a value that is the average level detected in healthy persons plus a value of several times (for example, twice) the standard deviation. Furthermore, the cut-off level can be appropriately changed to another level in accordance with ethnicity, the region in which patients live, and the like.

According to the method of determining a lung cancer treatment of the present invention, it can be determined whether or not treatment including use of an EGFR inhibitor will be effective simply based on the amount of KL-6.

The phrase “treatment including use of an EGFR inhibitor will be effective” means that the patient with lung cancer will obtain some remedial effects from the treatment including use of the EGFR inhibitor. The remedial effects may be confirmed by an increased survival rate, a decrease in the progression rate of the cancer or the like. In other words, the effectiveness of the EGFR inhibitor that will be administered to the patient may be determined in advance by the present invention. That is, the present invention includes determination of the effectiveness of the EGFR inhibitor on a patient with lung cancer.

The second embodiment of the present invention is a method of determining the effectiveness of an agent for treatment of lung cancer, and the method comprises measuring an amount of KL-6 in a sample of body fluid from the subject (a measuring step), and determining in advance the effectiveness of an EGFR inhibitor on the subject should the EGFR inhibitor be administered as the agent for treatment of lung cancer, based on the amount of KL-6 measured, (a determining step).

Matters relating to samples of body fluid, KL-6 and lung cancer with respect to the method of determining the effectiveness of the agent for treatment of lung cancer may be the same as in the descriptions for the method of determining lung cancer treatment described above.

The measuring step of the method of determining the effectiveness of the agent for treatment of lung cancer can be carried out in the same manner as the measuring step of the method of determining a lung cancer treatment described above.

In the determining step of the method of determining the effectiveness of the agent for treatment of lung cancer, the effectiveness of an EGFR inhibitor as the agent for treatment of lung cancer is determined. That is, in the method of determining the effectiveness, it may be predicted whether or not the EGFR inhibitor used in treatment of lung cancer will be effective on, for example, survival time, particularly, progression free survival time and overall survival time after the EGFR inhibitor treatment, prior to administering the agent.

For example, when the amount of KL-6 in the body fluid sample of the subject is equal to or higher than a reference level set in order to distinguish between a healthy person and a patient with lung cancer, it can be determined that the agent for treatment of lung cancer may not be effective on the patient (the effectiveness of the EGFR inhibitor will be low). On the other hand, when the amount of KL-6 in the body fluid sample of the subject is lower than the reference level, it can be determined that treatment using the EGFR inhibitor may be expected to be effective (the effectiveness of the EGFR inhibitor will be high).

The reference level used in the determining step may be the same reference level described in the above method of determining a lung cancer treatment.

According to the method of determining the effectiveness of an agent for treatment of lung cancer of the present invention, it can be determined whether or not treatment using an EGFR inhibitor will be effective if the EGFR inhibitor is administered, simply based on the amount of KL-6.

The present invention will be described in further detail using the following examples; however, the present invention is not limited thereto.

### EXAMPLES

### 1. Patients and Methods

1.1. Patients

We retrospectively reviewed the medical records of 41 NSCLC patients who underwent gefitinib monotherapy at Okayama University Hospital between September 2002 and September 2005. All patients fulfilled the following eligibility criteria enrolled consecutively in this study: 1) NSCLC patients who underwent gefitinib monotherapy after failure of at least one prior cytotoxic chemotherapy, 2) those whose serum KL-6 level was measured within one month before the initiation of gefitinib treatment. Potential factors linked to survival such as age, gender, performance status, smoking history, tumor histology, number of prior chemotherapy regimen, and presence of distant metastasis were also reviewed for each patient. Pretreatment interstitial lung disease was diagnosed using a high resolution computed tomography scan of the chest by diagnostic radiologists blinded to any clinical information.

1.2. Treatment

Gefitinib was orally administered at a daily dose of 250 mg and it was continued until the occurrence of either progressive disease (PD), unacceptable toxicity, or patient withdrawal. The tumor response was assessed as complete response (CR), partial response (PR), no change (NC), or progressive disease (PD) according to the World Health Organization Criteria [19].

1.3. Measurement of the Serum KL-6 Levels

The serum KL-6 levels were measured by an electrochemiluminescence immunoassay or enzyme immunoassay (Picolumi KL-6 or Eitest KL-6, Sanko Junyaku, Tokyo, Japan) within one month before the initiation of gefitinib treatment. The cut-off level of KL-6 was set at 500 U/ml based on the levels of healthy individuals as reported previously [16].

1.4. DNA Extraction and Direct Sequencing

Genomic DNAs were extracted from frozen or paraffin embedded specimens and the mutational status of the EGFR tyrosine kinase domain (through exons 18 to 21) was examined by the direct sequence method as described previously [8, 9]. In brief, the PCR amplification for extracted DNAs was done using HotStarTaq DNA polymerase (Qiagen Inc., Valencia, Calif.) with the specific primers and PCR conditions. PCR products were incubated using ExoSAP-IT (Amersham Biosciences Corp., Piscataway, N.J.) and then it was sequenced directly using the Applied Biosystems PRISM dye terminator cycle sequencing method (Perkin-Elmer Corp., Foster City, Calif.) with ABI PRISM 3100 Genetic Analyzer (Applied Biosystems, Foster City, Calif.).

1.5. Statistical Analysis

The associations between various clinical factors were evaluated using either the χ² test or the Fisher's exact test. The progression-free survival time after gefitinib treatment was defined as the time from the beginning of gefitinib treatment to the day of documented PD or death. The overall survival time was also defined as the time from the beginning of gefitinib treatment to death or the last follow-up day. The progression-free and overall survival times were both calculated using the Kaplan-Meier method; different groups were compared using the log-rank test. Associations of various factors with survival were evaluated using the backward stepwise Cox regression analysis. Differences were considered to be statistically significant if the p value was less than 0.05.

### 2. Results

2.1. Patient Characteristics

Table 1 summarizes the characteristics of the 41 NSCLC patients analyzed. The median age was 67 years (range, 34-82 years), and the majority of patients were male (61%), with a history of smoking (71%), and had an adenocarcinoma histology (85%). Approximately half of all patients (54%) received two or more prior types of chemotherapy before starting the gefitinib treatment. According to the TNM staging system, these recurrent cases were regarded as the following stages at the time of gefitinib treatment: IIIA, 4 (10%); IIIB, 5 (12%); and IV, 32 (78%). Ten patients (24%) had pretreatment interstitial lung diseases: namely 4 had radiation fibrosis; 4 had idiopathic pulmonary fibrosis; 1 had pneumoconiosis; and 1 had drug-induced pneumonitis. The serum KL-6 levels of the 41 patients ranged from 199 to 9080 U/ml (median, 550 U/ml), and 22 (54%) patients demonstrated elevated KL-6 levels (≧500 U/ml).

TABLE 1
Demographics of the 41 patients with NSCLC at the initiation of
gefitinib treatment

| | |
|---|---|
| Median age (range) | 67 (34-82) |
| Gender (male/female) | 25 (61%)/16 (39%) |
| Performance status (0-1/≧2) | 27 (66%)/14 (34%) |
| Smoking history (ever/never) | 29 (71%)/12 (29%) |
| Interstitial lung disease (yes/no) | 10 (24%)/31 (76%) |
| Prior chemotherapy (1/2/≧3) | 19 (46%)/13 (32%)/9 (22%) |
| Histology (Ad/Sq/others) | 35 (85%)/5 (12%)/1 (2%) |
| Stage (IIIA/IIIB/IV) | 4 (10%)/5 (12%)/32 (78%) |
| Median KL-6 (U/ml) (range) | 550 (199-9080) |
| Elevated KL-6 (yes/no) | 22 (54%)/19 (46%) |

NSCLC = non-small cell lung cancer,
Ad = adenocarcinoma,
Sq = squamous cell carcinoma.

2.2. Treatment Outcomes

All patients were assessable for their response to gefitinib. One CR and ten PRs, were observed, which resulted in an overall objective response rate of 26.8%. NC and PD were observed in 20 and 10 patients, respectively. Early discontinuance of gefitinib (less than 2 weeks) was observed in 3 (7%) patients because of rapid tumor progression. The median follow-up time for the surviving patients was 20.6 months. The median progression-free survival time, median overall survival time, 1-year, and 2-year survival rates for 41 patients were 4.7 months, 13.9 months, 61.0%, and 29.8%, respectively. During the study period, neither severe interstitial lung disease nor treatment-related death occurred due to the gefitinib treatment.

2.3. Association Between the Serum KL-6 Levels and Clinical Factors

We investigated the relationship between the serum KL-6 levels and various clinical factors (Table 2). A statistically significant association was observed between the elevated KL-6 levels and a poor performance status (≧2) (p=0.022). However, no other factors, including gender, smoking history, or tumor histology were associated with the pretreatment serum KL-6 levels. Although the patients with pretreatment interstitial lung diseases tended to have increased serum KL-6 levels, the difference did not reach statistical significance in this small retrospective study (70% versus 48%, p=0.292). There was also no difference in the response to gefitinib between patients with or without elevated serum KL-6 levels (23% versus 32%, p=0.725).

TABLE 2
Association between an elevated KL-6 level and clinical factors

| | | | | Proportion | |
|---|---|---|---|---|---|
| | | | | of patients | |
| | | Patients with | Patients with | with elevated | |
| Factors | | normal KL-6 | elevated KL-6 | KL-6 | p* |
| Total (n = 41) | | 19 | 22 | 54% | |
| Age | 75< | 15 | 18 | 55% | >0.999 |
| | ≧75 | 4 | 4 | 50% | |
| Gender | male | 9 | 16 | 64% | 0.120 |
| | female | 10 | 6 | 38% | |
| Performance status | 0-1 | 16 | 11 | 41% | 0.046 |
| | ≧2 | 3 | 11 | 79% | |
| Smoking history | ever | 12 | 17 | 59% | 0.493 |
| | never | 7 | 5 | 42% | |
| Interstitial lung disease | yes | 3 | 7 | 70% | 0.292 |
| | no | 16 | 15 | 48% | |
| Prior chemotherapy | 1-2 | 14 | 14 | 50% | 0.524 |
| | ≧3 | 5 | 8 | 62% | |
| Histology | Ad | 15 | 20 | 57% | 0.390 |
| | Non-Ad | 4 | 2 | 33% | |
| Disease stage | III | 4 | 6 | 56% | 0.727 |
| | IV | 15 | 16 | 53% | |
| Response to gefitinib | yes | 6 | 5 | 45% | 0.725 |
| | no | 13 | 17 | 57% | |

*Fisher's exact test, Abbreviations: Ad = adenocarcinoma.

2.4. Association Between the Survival After Gefitinib Treatment and Clinical Factors

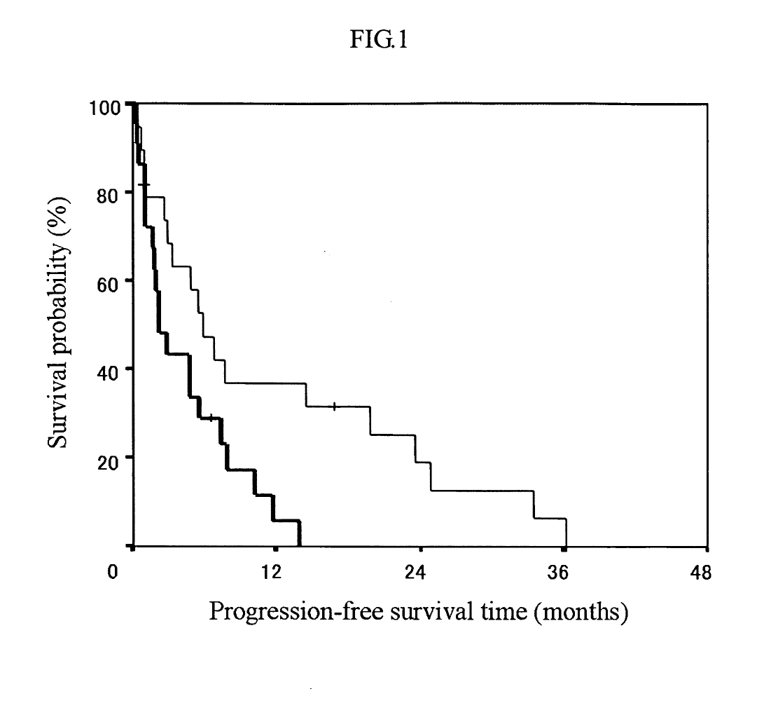

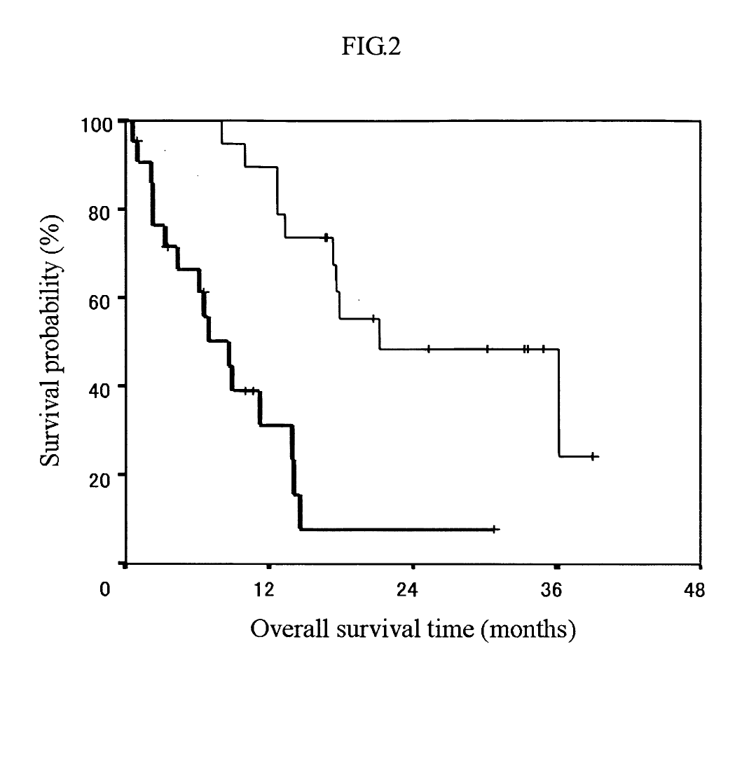

A univariate analysis demonstrated that patients with elevated KL-6 levels had a significantly shorter progression-free and overall survival than those demonstrating a normal range of KL-6 (median progression-free survival time: 2.1 months versus 5.8 months, p=0.0220; median overall survival time: 8.5 months versus 21.1 months, p=0.0001) (FIGS. 1 and 2). The following clinical factors were also associated with unfavorable outcomes; male (p=0.0011), smokers (p=0.0001), the presence of pretreatment interstitial lung disease (p=0.0033) in the progression-free survival, poor performance status (>2) (p=0.047) and the presence of pretreatment interstitial lung disease (p=0.0001) in the overall survival. To rule out any potential confounding factors, we conducted a multivariate analysis for the possible variables related to survival after gefitinib treatment. As a result, an elevated KL-6 level was thus found to be an independent adverse prognostic factor for both a progression-free survival (hazard ratio: 2.278, p=0.040) and overall survival (hazard ratio: 4.858, p=0.002) after gefitinib treatment in the patients with NSCLC (Tables 3 and 4).

TABLE 3
Multivariate analysis for the progression-free survival after
gefitinib treatment in patients with NSCLC

| Factors | HR | 95% CI | p* |
|---|---|---|---|
| Elevated KL-6 (yes/no) | 0.439 | 0.200-0.965 | 0.040 |
| Interstitial lung disease (yes/no) | | | N.S |
| Performance status (0-1/≧2) | | | N.S |
| Histology (Ad/Non-Ad) | 3.102 | 1.161-8.284 | 0.024 |
| Gender (male/female) | | | N.S |
| Smoking history (ever/never) | 0.179 | 0.066-0.483 | 0.001 |

*backward stepwise Cox-regression analysis,
Abbreviations:
NSCLC = non-small cell lung cancer,
Ad = adenocarcinoma,
HR = hazard ratio,
CI = confidence interval,
N.S = not significant.

TABLE 4
Multivariate analysis for the overall survival after gefitinib
treatment in patients with NSCLC

| Factors | HR | 95% CI | p* |
|---|---|---|---|
| Elevated KL-6 (yes/no) | 0.198 | 0.068-0.579 | 0.003 |
| Interstitial lung disease (yes/no) | 0.225 | 0.084-0.607 | 0.003 |
| Performance status (0-1/≧2) | 3.042 | 1.263-7.325 | 0.013 |
| Histology (Ad/Non-Ad) | 2.871 | 0.973-8.476 | 0.056 |
| Gender (male/female) | | | N.S |
| Smoking history (ever/never) | | | N.S |

*backward stepwise Cox-regression analysis,
Abbreviations:
NSCLC = non-small cell lung cancer,
Ad = adenocarcinoma,
HR = hazard ratio,
CI = confidence interval,
N.S = not significant.

2.5. Association Between Serum KL-6 Levels and EGFR Mutation

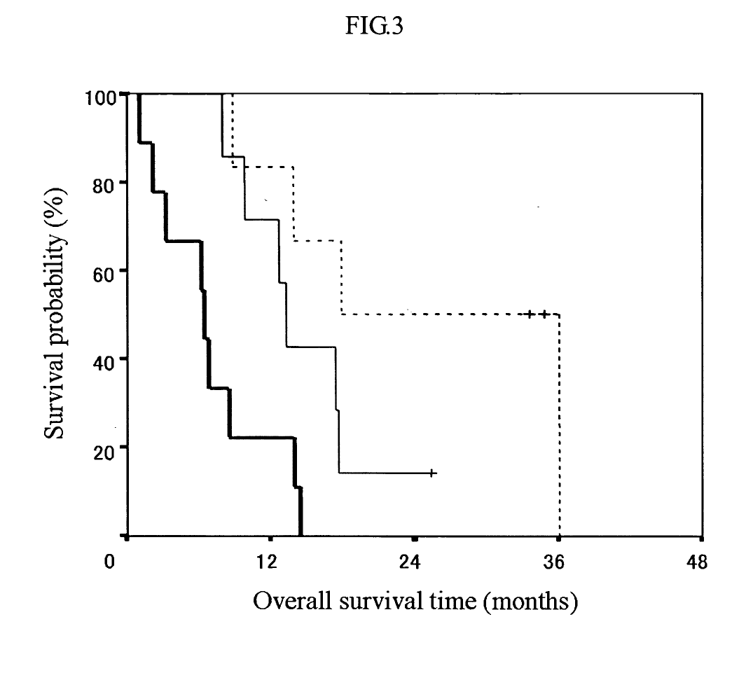

Of the 41 patients, 22 patients (54%) were available to provide tumor samples to analyze the presence of EGFR mutations. Among these 22 patients, six patients (27%) had EGFR mutations in their tumor (3 in exon 21, 2 in exon 19, and 1 in exon 18), whereas the remaining 16 (73%) had wild-type EGFR. An EGFR mutation was significantly associated with a higher response rate, but not with the serum KL-6 levels. As expected, an EGFR mutation was found to be able to distinguish favorable and unfavorable prognostic groups; there was a significant difference in the overall survival between the patients with an EGFR mutation and those with wild-type EGFR (17.8 months versus 8.5 months, p=0.0177). Interestingly, the serum KL-6 levels further stratify the survival after gefitinib treatment in patients with wild-type EGFR. The median survival time in patient with or without elevated serum KL-6 levels was 6.5 months versus 13.3 months, respectively (p=0.0194), despite the limited number of patients (FIG. 3).

### 3. Discussion

In this study, we found a close relationship between the serum levels of KL-6 and the survival time in NSCLC patients treated with gefitinib. Patients with high serum KL-6 levels showed a significantly shorter progression free survival time than those with normal serum KL-6 levels. These results held true for the overall survival even when EGFR mutations are taken into account. This easily assessable and serological biomarker might therefore provide additional valuable information for predicting the life expectancy for NSCLC patients when we intend to treat them with gefitinib.

Among the various predictors for the clinical outcomes of gefitinib treatment, activating EGFR mutations have been considered to be one of the most reliable prognostic factors ever studied. Based on its high predictability for response and survival, a number of phase II studies for NSCLC patients with EGFR mutations have been performed to evaluate the effectiveness of gefitinib, and response rates have been reported to range from 75% to 81% [20-23]. These emerging data are indeed promising. However, patients harboring EGFR mutations account for a small fraction of the whole NSCLC; the prevalence of EGFR mutations has been reported to be as high as 30% in Japanese, and around 10% in Caucasians [10]. Furthermore, we actually experienced NSCLC patients who had wild-type EGFR gene but nevertheless achieved a favorable outcome after the initiation of gefitinib treatment [6-9]. Considering these circumstance, it is clinically meaningful that KL-6 further stratified the survival in NSCLC patients without EGFR mutations because they are currently considered to be unlikely to benefit from gefitinib.

Although the exact reason why an elevated KL-6 level is inversely associated with the survival after gefitinib treatment in patients with NSCLC is still unknown, some explanations can be considered. In a previous study of hepatocellular carcinoma, the serum KL-6 levels were reported to represent the level of MUC 1 expression, thus suggesting that an elevation of KL-6 in the serum may be translated into a higher expression of MUC1 in NSCLC patients [24]. In addition, increased MUC1 expression inhibits the E-cadherin expression [25] that has been shown to confer sensitivity to gefitinib in lung cancer cell lines [26]. These findings suggest that KL-6 plays a different role in gefitinib treatment, and it might modulate its clinical efficacy through some possible interaction between the MUC1 and E-cadherin expressions. Another possibility is that an elevated KL-6 level represents a more aggressive behavior of the tumor, thus resulting in a diminished outcomes regardless of the therapy. It is known that an overexpression of MUC1 is associated with tumor invasiveness, metastasis, and immune escape [25]. A recent study demonstrated that high serum level of KL-6 was significantly associated with the poor survival of lung adenocarcinoma patients receiving various treatments including surgery, radiotherapy, and chemotherapy [27]. The KL-6 levels may therefore adversely affect the natural history or any treatment course in NSCLC. Since increased serum KL-6 levels were frequently observed, not only in patients with NSCLC, but also in those with interstitial lung diseases, it is still unclear whether the serum KL-6 levels could directly reflect the tumor biology of NSCLC. We can not completely rule out the possibility that the serum KL-6 levels simply represent the activity of interstitial lung diseases accompanied with primary lung cancer and thus the poor clinical outcome in patients with high serum KL-6 levels are simply considered to be attributable to a deterioration of interstitial lung diseases rather than NSCLC. However, it is also the fact that interstitial lung diseases were not always detected in all patients with high KL-6 levels throughout their clinical course. Additionally, even multivariate analyses using data that excluded these 4 patients with idiopathic pulmonary fibrosis from the study demonstrated that serum KL-6 levels was still significant adverse prognostic factor (progression-free survival; hazard ratio=0.426, p=0.048, overall survival; hazard ratio=0.162,p=0.003). These findings indicate that serum KL-6 levels could reflect the tumor biology of NSCLC rather than interstitial lung disease in the gefitinib treatment. Further evaluation is thus required to determine the clinical significance of KL-6 in lung cancer patients in the future.

In the present study, we demonstrated that elevated serum KL-6 levels significantly correlated with both poorer progression-free and overall survivals, but not with response to gefitinib. This discordant result between response and survival data may simply arise from our small sample size. As another explanation, disease stabilization as well as objective response would be an indicator of a benefit from gefitinib treatment, based on the recently reported role of disease stabilization in survival advantage [28], and thus, high response rate might not always correlate with survival prolongation by gefitinib treatment. Further evaluation is warranted to resolve these questions.

### REFERENCES

[1] Fukuoka M, Yano S, Giaccone G, Tamura T, Nakagawa K, Douillard J Y, et al. Multi-institutional randomized phase II trial of gefitinib for previously treated patients with advanced non-small-cell lung cancer (The IDEAL 1 Trial). J Clin Oncol. 2003, 21: 2237-46.
[2] Kris M G, Natale R B, Herbst R S, Lynch T J Jr, Prager D, Belani C P, et al. Efficacy of gefitinib, an inhibitor of the epidermal growth factor receptor tyrosine kinase, in symptomatic patients with non-small cell lung cancer: a randomized trial. JAMA. 2003, 290: 2149-58.
[3] Thatcher N, Chang A, Parikh P, Rodrigues Pereira J, Ciuleanu T, von Pawel J, et al. Gefitinib plus best supportive care in previously treated patients with refractory advanced non-small-cell lung cancer: results from a randomised, placebo-controlled, multicentre study (Iressa Survival Evaluation in Lung Cancer). Lancet. 2005, 366: 1527-37.
[4] Lee D H, Han J Y, Yu S Y, Kim H Y, Nam B H, Hong E K, et al. The role of gefitinib treatment for Korean never-smokers with advanced or metastatic adenocarcinoma of the lung: a prospective study. J Thorac Oncol. 2006, 1: 965-71
[5] Yang C, Yu C, Chen K, Lin Z, Kuo S, Hwang T, et al. A phase II study of gefitinib as first line treatment for good performance advanced or metastatic non-small cell lung cancer in East Asian patients. Proc Am Soc Clin Oncol 2006; 24: (Abstr 7167).
[6] Mitsudomi T, Kosaka T, Endoh H, Horio Y, Hida T, Mori S, et al. Mutations of the epidermal growth factor receptor gene predict prolonged survival after gefitinib treatment in patients with non-small-cell lung cancer with postoperative recurrence. J Clin Oncol. 2005, 23: 2513-20.
[7] Hotta K, Kiura K, Toyooka S, Takigawa N, Soh J, Fujiwara Y, et al. Clinical significance of epidermal growth factor receptor gene mutations on treatment outcome after first-line cytotoxic chemotherapy in Japanese patients with non-small cell lung cancer. J Thorac Oncol. 2007, 2: 632-7.
[8] Tokumo M, Toyooka S, Kiura K, Shigematsu H, Tomii K, Aoe M, et al. The relationship between epidermal growth factor receptor mutations and clinicopathologic features in non-small cell lung cancers. Clin Cancer Res. 2005, 11: 1167-73.
[9] Ichihara S, Toyooka S, Fujiwara Y, Hotta K, Shigematsu H, Tokumo M, et al. The impact of epidermal growth factor receptor gene status on gefitinib-treated Japanese patients with non-small-cell lung cancer. Int J Cancer. 2006 (in press)
[10] Johnson B E, Janne P A. Epidermal growth factor receptor mutations in patients with non-small cell lung cancer. Cancer Res. 2005, 65: 7525-9.
[11] Cappuzzo F, Hirsch F R, Rossi E, Bartolini S, Ceresoli G L, Bemis L, et al. Epidermal growth factor receptor gene and protein and gefitinib sensitivity in non-small-cell lung cancer. J Natl Cancer Inst. 2005, 97: 643-55.
[12]Takano T, Ohe Y, Sakamoto H, Tsuta K, Matsuno Y, Tateishi U, et al. Epidermal growth factor receptor gene mutations and increased copy numbers predict gefitinib sensitivity in patients with recurrent non-small-cell lung cancer. J Clin Oncol. 2005, 23: 6829-37.
[13]Pao W, Wang T Y, Riely G J, Miller V A, Pan Q, Ladanyi M, et al. KRAS mutations and primary resistance of lung adenocarcinomas to gefitinib or erlotinib. PLoS Med. 2005, 2: e 17.
[14] Giaccone G, Janmaat M, Ruiz M G, Rodriguez J, LeChevalier T, Thatcher N, et al. EGFR mutations do not accurately predict response to erlotinib in first-line monotherapy treatment of advanced non-small-cell lung cancer. Lung Cancer. 2005; 49: S244 (P-485).
[15] Kohno N, Kyoizumi S, Awaya Y, Fukuhara H, Yamakido M, Akiyama M. New serum indicator of interstitial pneumonitis activity. Sialylated carbohydrate antigen KL-6. Chest. 1989, 96: 68-73.
[16]Kohno N, Akiyama M, Kyoizumi S, Hakoda M, Kobuke K, Yamakido M. Detection of soluble tumor-associated antigens in sera and effusions using novel monoclonal antibodies, KL-3 and KL-6, against lung adenocarcinoma. Jpn J Clin Oncol. 1988, 18: 203-16.
[17] Hirasawa Y, Kohno N, Yokoyama A, Inoue Y, Abe M, Hiwada K. KL-6, a human MUC 1 mucin, is chemotactic for human fibroblasts. Am J Respir Cell Mol Biol. 1997, 17: 501-7.
[18] Guddo F, Giatromanolaki A, Koukourakis M I, Reina C, Vignola A M, Chlouverakis G, et al. MUC 1 (episialin) expression in non-small cell lung cancer is independent of EGFR and c-erbB-2 expression and correlates with poor survival in node positive patients. J Clin Pathol. 1998, 51: 667-71.
[19] Miller A B, Hoogstraten B, Staquet M, Winkler A. Reporting results of cancer treatment. Cancer. 1981, 47: 207-14.
[20] Inoue A, Suzuki T, Fukuhara T, Maemondo M, Kimura Y, Morikawa N, et al. Prospective phase II study of gefitinib for chemotherapy-naive patients with advanced non-small-cell lung cancer with epidermal growth factor receptor gene mutations. J Clin Oncol. 2006, 24: 3340-6.
[21] Sutani A, Nagai Y, Udagawa K, Uchida Y, Koyama N, Murayama Y, et al. Gefitinib for non-small-cell lung cancer patients with epidermal growth factor receptor gene mutations screened by peptide nucleic acid-locked nucleic acid PCR clamp. Br J Cancer. 2006, 95: 1483-9.
[22] Okamoto I, Kashii T, Urata Y, Hirashima T, Kudoh S, Ichinose Y, et al. EGFR mutation-based phase II multicenter trial of gefitinib in advanced non-small cell lung cancer (NSCLC) patients (pts): Results of West Japan Thoracic Oncology Group trial (WJTOGO403). Proc Am Soc Clin Oncol 2006; 24: (Abstr 7073).
[23] Sunaga N, Yanagitani N, Kaira K, Tomizawa Y, lijima H, Otani Y, et al. Phase II study of the efficacy of gefitinib in patients with non-small cell lung cancer with the EGFR mutations. Proc Am Soc Clin Oncol 2006; 24: (Abstr 7183).
[24] Moriyama M, Matsumura H, Watanabe A, Nakamura H, Arakawa Y, Oshiro S, et al. Detection of serum and intrahepatic KL-6 in anti-HCV positive patients with hepatocellular carcinoma. Hepatol Res. 2004, 30: 24-33.
[25]Hollingsworth M A, Swanson B J. Mucins in cancer: protection and control of the cell surface. Nat Rev Cancer. 2004, 4: 45-60.
[26]Witta S E, Gemmill R M, Hirsch F R, Coldren C D, Hedman K, Ravdel L, et al. Restoring E-cadherin expression increases sensitivity to epiderrnal growth factor receptor inhibitors in lung cancer cell lines. Cancer Res. 2006, 66: 944-50.
[27] Inata J, Hattori N, Yokoyama A, Ohshimo S, Doi M, Ishikawa N, et al. Circulating KL-6/MUC1 mucin carrying sialyl Lewisa oligosaccharide is an independent prognostic factor in patients with lung adenocarcinoma. Int J Cancer. 2007, 120: 2643-9.
[28] Hotta K, Matsuo K, Ueoka H, Kiura K, Tabata M, Harita S, et al.. Continued gefitinib treatment after disease stabilisation prolongs survival of Japanese patients with non-small-cell lung cancer: Okayama Lung Cancer Study Group experience. Ann Oncol. 2005, 16:1817-23.

All publications, patent applications, and technical standards mentioned in this specification are herein incorporated by reference to the same extent as if such individual publication, patent application, or technical standard was specifically and individually indicated to be incorporated by reference. It will be obvious to those having skill in the art that many changes may be made in the above-described details of the preferred embodiments of the present invention. The scope of the invention, therefore, should be determined by the following claims.

What is claimed is:

1. A method of determining a lung cancer treatment, the method comprising:
measuring an amount of KL-6 in a sample of body fluid from a subject, and determining the treatment based on the amount of KL-6 measured.

2. The method according to claim 1, wherein the treatment includes use of an EGFR (epidermal growth factor receptor) inhibitor as an agent for treatment of lung cancer.

3. The method according to claim 2, wherein the EGFR inhibitor is an EGFR tyrosine kinase inhibitor.

4. The method according to claim 2, wherein the EGFR inhibitor is gefitinib.

5. The method according to claim 2, wherein the lung cancer is non-small cell lung cancer (NSCLC).

6. The method according to claim 1, wherein the determining is conducted so as to select the treatment when the amount of KL-6 in the body fluid sample of the subject is not higher than a reference level.

7. The method according to claim 2, wherein the determining is conducted so as to select the treatment including use of the EGFR inhibitor when the amount of KL-6 in the body fluid sample of the subject is not higher than a reference level.

8. The method according to claim 1, wherein the body fluid sample is at least one selected from the group consisting of blood, serum, plasma and lymphatic fluid.

9. The method according to claim 1, wherein the measuring of the amount of KL-6 includes use of an anti-KL-6 antibody.

10. The method according to claim 1, wherein the measuring of the amount of KL-6 is a measurement by immunoassay including use of an anti-KL-6 antibody and a labeled antibody.

11. A method of determining the effectiveness of an agent for treatment of lung cancer on a subject, the method comprising:
measuring an amount of KL-6 in a sample of body fluid from the subject, determining the effectiveness of an EGFR inhibitor on the subject should the EGFR inhibitor be administered as the agent for treatment of lung cancer, based on the amount of KL-6 measured.

12. The method according to claim 11, wherein the EGFR inhibitor is an EGFR tyrosine kinase inhibitor.

13. The method according to claim 11, wherein the EGFR inhibitor is gefitinib.

14. The method according to claim 11, wherein the lung cancer is non-small cell lung cancer.

15. The method according to claim 11, wherein the determining is conducted so as to determine that the effectiveness of the EGFR inhibitor will be low when the amount of KL-6 in the body fluid sample of the subject is equal to or higher than a reference level.

16. The method according to claim 11, wherein the body fluid sample is at least one selected from the group consisting of blood, serum, plasma and lymphatic fluid.

17. The method according to claim 11, wherein the measuring of the amount of KL-6 includes use of an anti-KL-6 antibody.

18. The method according to claim 11, wherein the measuring of the amount of KL-6 is a measurement by immunoassay including use of an anti-KL-6 antibody and a labeled antibody.
